(19) United States
(12) Patent Application Publication
(10) Pub. No.: US 2018/0132470 A1
(43) Pub. Date: May 17, 2018

(54) CONTROLLED STAND-ALONE MATRIX SYSTEM FOR THE CONTROLLED DISTRIBUTION OF ACTIVE SUBSTANCES

(71) Applicant: AB7 Innovation S.A.S.U., DEYME (FR)

(72) Inventors: René CHELLE, Grepiac (FR); David NGUYEN, Toulouse (FR); Arnaud VILBERT, Baziège (FR); Sylvain AUGIER, Toulouse (FR)

(21) Appl. No.: 15/564,380

(22) PCT Filed: Apr. 8, 2016
(86) PCT No.: PCT/FR2016/000071

§ 371 Date: Oct. 4, 2017

(30) Foreign Application Priority Data
Apr. 10, 2015 (FR) 15 00747

(51) Int. Cl. A01M 1/20; A61L 9/03; A01N 25/10; A01N 25/18

(52) U.S. Cl. CPC A01M 1/2077; A01N 25/18; A01N 25/10; A61L 9/03

(57) ABSTRACT

The invention relates to a controlled stand-alone matrix system (1) for the controlled and variable distribution of at least one active substance solely at precise appropriate moments, characterised in that it comprises a polymer matrix (2) loaded with the active substance, at least one heating element (4) embedded in the loaded matrix (2), and an electricity generator (5) connected to an electronic control card in turn connected to the heating element (4), the electricity supplied to same by the generator (5) being controlled by the card which is housed in an electronic box (3).

This invention concerns the field of systems enabling the distribution of active substances to treat and/or control targets.

To be precise, this invention relates to a controlled matrix system for the distribution of active substances in a controlled quantity and solely at appropriate moments, for human and/or animal use, so that the substance released responds perfectly to an effectiveness requirement pre-defined and/or known depending on the intended target.

Devices designed to diffuse active substances such as pheromones, insecticides, perfume and deodorizers are known. Generally, they can be classified into three main categories:
reservoir device for which the active substance in liquid solution is loaded into a reservoir. Said substance is diffused into the atmosphere either by migration through the semi-permeable polymer wall of said reservoir or by spraying in the form of droplets;
matrix device in which the active substance solubilized in a compatible solvent is incorporated into a polymer matrix of various forms;
micro-particle device in which the active substance is pre-loaded into an absorbent micro-support for an application by spraying after dispersion in a carrier fluid.

This invention is classified in the category of matrix devices associated with other components so that the assembly constitutes a system.

More particularly, the subject matter of this invention relates to a controlled stand-alone matrix system for the distribution at controlled speed and at precise appropriate moments of active substances incorporated into a polymer matrix. Said system comprises at least three essential components:
a polymer matrix loaded with a composition containing at least one active substance;
a heating element embedded in said loaded matrix, said element being capable of uniformly heating said matrix to trigger the release of said active substance, and
an electricity generator supplying an electronic control card housed in an electronic box.

Devices comprising a hydrophobic polymer matrix for the distribution of active substances are also known. Said devices can in particular be in the form of bracelets, necklaces, tablets or any other form. Such devices only enable a continuous passive diffusion at ambient temperature of the active substances incorporated therein. This diffusion starts as soon as the pack is opened and cannot be stopped unless a new sealed pack is inserted, which requires the user to perform additional manipulations. The choice of formulation vectors of said active substances, that of the polymers for the matrix as well as the dimensions of the final device constitute the only way of controlling the release of these substances. Now, continuous diffusion is not always suitable for certain very specific treatments, for example when a localized diffusion of active substance is required solely at precise moments. Moreover, some polymer matrices that perform passive diffusion at ambient temperature sequester a considerable quantity, up to 50% by weight, of active substance at the end of its presumed use. In fact, the Applicant has observed that some non-volatile or low-volatile active molecules remain sequestered in the matrix, however, despite their optimal formulation by means of good vectors. Furthermore, some medical devices require that only part of the full release potential is used in order to be sure to use a kinetics zone where the dose is constant and at a level above the threshold of effectiveness. This operating mode entails a considerable waste of active substances and, on disposal, these devices still contain some of their active substances.

Although the Applicant has already developed passive-diffusion polymer-matrix devices in the form of tablets or bracelets, as described in patents FR2901096, FR2951645 and FR2959396, no known device based on a polymer matrix allows any type of active substance to be delivered at a controlled speed solely at precise appropriate moments. Moreover, no known matrix device is capable of releasing the active substances at a controlled speed so that the released quantity perfectly meets an effectiveness requirement pre-defined and/or known.

In order to improve the operation of passive-diffusion matrix devices, other components have been added so that the assembly constitutes a system. Some of the added components include plate- or heating-resistor heating elements applied to the polymer matrix. In this regard, the prior art is divided into two clearly distinct categories of matrix system:
either, said heating elements are applied to plant-origin hydrophilic polymer matrices loaded with active substance,
or, said heating elements are associated with hydrophobic-polymer matrices, but these are not loaded with active substance.

Matrix systems are therefore known comprising heating elements to promote the diffusion of active substances that are volatile in almost all cases.

For example, patent FR2722368 discloses a wick-insecticide diffuser provided with a battery for aerial diffusion by volatility for about 12 hours. The diffuser comprises a radiating plate onto which is placed a cellulosic-matrix pad impregnated with pyrethroid insecticides. The surface of the said plate is heated to a temperature below 130° C. to preserve the insecticide. The heating element consists in an organic thermistor formed by a mixture of polyethylene resin or a polypropylene and carbon resin. But, the matrix used is hydrophilic and there is no pre-programmed electronic control card in the diffuser to control the battery in order to obtain a controlled quantity of insecticides for better effectiveness.

In the same vein, patents FR2431256, EP1108358, EP1101500, EP1579762, EP1372161 and FR2322546 describe a system comprising a heating element and a hydrophilic wick immerged in a reservoir containing a liquid composition of insecticide and/or perfume for aerial diffusion by volatility.

Patent BE1001662 describes a device for aerial diffusion by volatility of insecticides and deodorizers comprising electrical heating elements in a ceramic plate inside which is mounted an electrical resistor, said plate being designed to support and heat a tablet loaded with insecticides. In order to operate the device, said tablet is placed on a plate then the electric assembly is switched on. But this document makes no mention of the physico-chemical nature of the matrix and there is no pre-programmed electronic control card to obtain an effective quantity of insecticides.

Patent application US2007/257016 claims a device with an electric battery for the diffusion of a volatile insecticide substance in the form of a vapor cloud. Said insecticide substance is impregnated in a porous or fibrous matrix such as woven or non-woven fibers. An electrical resistor arranged outside said matrix is placed in contact therewith to cause the diffusion of the insecticide. In the same vein, patent U.S. Pat. No. 6,309,986 describes a system comprising a layered cellulosic matrix impregnated with volatile insecticide. A metal heating resistor is arranged on the layer containing the insecticide so as to heat the latter and cause the diffusion of the insecticide. But, the matrix is hydrophilic and the battery is not controlled by any pre-programmed electronic card. Moreover, only volatile substances are compatible with these two systems.

Hydrophobic matrices not loaded with active substance are disclosed in patent FR2888715 which describes a device designed to protect plantations against freezing, said device being in the form of a multi-layered plastic film of which at least one of the layers comprises heating elements. Said heating elements are distributed in modules powered by an electrical source. But, the multi-layer film of the system is not loaded with any active substance. Along the same lines, international application WO2005039240 discloses a device with multi-layer thermoplastic polymer film meeting heating requirements over large areas, particularly in viticulture. Said device comprises electrical conductor means to be able to cause by Joule effect heating at the surface of said film. But, the multi-layer film of the system is not loaded with any active sub stance.

In a more complex design, patent EP1629696 discloses a flexible garment heater comprising laminated layers of which at least one comprises heating elements. Said heating elements are connected to a battery and are incorporated into the fabric of which certain fibers are metalized. One of the layers is an adhesive film of impermeable thermoplastic polymers. Capsules containing volatile active substances are fixed onto the fibers. Said volatile substances are released by the capsules bursting due to the effect of the heat. But the matrix is layered and the active substances must be encapsulated. Moreover, said substances are not incorporated directly into the layer of thermoplastic polymers and there is no electronic control card to control the battery.

Despite all of the solutions proposed by the prior art, there is a need to provide a controlled stand-alone matrix system for the controlled distribution of any type of active substance, namely liquid and solid, volatile or non-volatile. Said system basically allows said active substances incorporated in a polymer matrix to be released on command by heating at precise appropriate moments in sufficient quantity to confer to a given treatment a better effectiveness with respect to an intended target. The aim of the present invention is to overcome the drawbacks encountered in the prior art.

One aim of the present invention is to provide a stand-alone matrix system controlled by a programmed electronic card for the controlled and modulatable distribution of at least one active substance in a pre-determined sufficient quantity solely at precise appropriate moments in order to confer on a treatment a better effectiveness depending on the intended target, said target comprising human, animal, insects or an open environment, particularly a field, or closed environment, particularly a room or a dwelling.

Another aim of the present invention is to propose a matrix system capable of distributing at least one active substance in a precisely determined quantity while minimizing the passive diffusion at ambient temperature of said active substance from the polymer matrix but while optimizing the release of the active substance solely at precise appropriate moments.

Another aim of the present invention is to propose a system within which the polymer matrix releases almost all of the active substance that it contains at the end of use of the system, unlike a passive diffusion polymer matrix device which keeps more than 10% of said substance sequestered at the end of its use. This allows the entire capacity of the reservoir to be exploited, which enables the matrix to be used for extended periods of time.

Another aim of the present invention is to provide a controlled stand-alone matrix system for the controlled distribution of non-volatile active substances at ambient temperature.

Yet another aim of the present invention is to provide a controlled stand-alone matrix system for the controlled and simultaneous or staggered distribution of several identical and/or different active substances, the multiple matrices being loaded with active substances and capable of being installed in series, each of the matrices being loaded with an identical or different active substances.

Yet another aim of the present invention is to provide a method that uses the matrix system according to claim 1 to effectively treat a target located in the same place or in spatially different places solely at precise appropriate moments by sequential or continuous diffusion of a previously-determined sufficient quantity of at least one active substance.

In the context of the invention, “electronic control box” means an assembly comprising at least one programmed electronic control card taking into account at least three essential parameters, namely heating intensity, heating time and heating frequency. The programming of the card is adapted so that it is capable of simultaneously handling at least three parameters depending on the intended target. Thanks to programming, the matrix system distributes the active substance according to a reference release profile. The system is started up automatically or by means of onboard or manual actuators such as switches and suchlike. Advantageously, in order to optimize and complete the reference release, said card is associated with at least one signal collector, possibly a stand-alone collector. Thus, said card controls the starting or stopping of the supply of electricity to the heating element by the generator, based on the signal sent by at least one collector via a line-based and/or wireless communication network (e.g. Wi-Fi®, Bluetooth or GSM), according to a structure of communication between collectors, network and stations, commonly called a Wireless Body Area Network (WBAN). The stations, either fixed such as computers, or mobile such as telephone and suchlike, are provided with a software interface enabling recognition of each of the line-based and/or wireless collectors as well as the signals that they emit. Similarly, the electrical supply via said card can be controlled by means of the above-mentioned wireless network. In this case, transmitters and receivers are arranged on the collector and on said card. The signal collector is a device capable of transforming the physico-chemical and physiological values into electrical signals. Said values are for example the temperature, pressure, pH, air, humidity, luminosity, presence, movement, heart rate, blood sugar, blood pressure or even respiration rate. Said collector can, for example, be a sensor, a probe, a detector or a combination thereof. More precisely, the electronic card comprises, in particular, a computing processor to process the command to activate the heating element, a memory unit designed to store the data gathered by the different collectors distributed, stand-alone and communicating, for their fusion by an algorithm in order to define a standard release profile, adapted to a treatment, depending on the target requirements. Said card forms an integral part of an electronic architecture, onboard or otherwise, enabling the reception, recording, surveillance and processing of signals from all of the system's collectors.

Thus, the electronic control card controls the system according to the invention by executing the functions chosen from among:
management of starting and stopping the electrical supply to the heating element(s) by the generator;
adjustment of the power developed by said generator;
adjustment of the duration, intensity and frequency of heating;
management of activation sequences;
management of the total duration of a treatment or an application;
management, if necessary, of starting, stopping and the power of a fan.

An “active matrix” means a polymer matrix in which is incorporated any type of active composition, be it liquid or solid, volatile or non-volatile. For the purpose of incorporation, said active matrix is dry.

A “controlled and modulatable” distribution means a mode of releasing the active substance from the matrix in a continuous and/or sequential way. A sequential distribution can be represented by a saw tooth diagram, or by a pulse diagram in which the amplitudes and/or frequencies are variable and depend on the three above-mentioned essential programming parameters.

A “minimized” released quantity of active substance means a quantity of active substance released, at ambient temperature by passive diffusion by the active matrix at rest with a content of less than 10%, preferably between 0.1% and 5% by weight in relation to the total quantity initially incorporated, for a period of 10 days.

An active substance that is “nearly all” released means the fact that at the end of use of the system the active matrix has release at least 90% of said substance in relation to the total quantity initially incorporated.

Thus the present invention relates to a controlled stand-alone matrix system for the controlled and modulatable distribution solely at precise appropriate moments of at least one active substance, said system comprising:
a non-layered polymer matrix loaded with said active substance, said matrix comprising at least one temperature probe,
at least one heating element embedded in the mass of said loaded matrix, said element being capable of heating evenly the entire said matrix so as to temporarily expand the polymer network in order to trigger the release of said active substance,
an electricity generator connected to an electronic control card, itself connected to said heating element, whose supply of electricity by said generator is controlled by said card housed in an electronic box, said card being connected, also, to said at least one temperature probe.

Surprisingly, knowing the effects of the action of heat on active matrixes, the research carried out by the inventors has shown that on certain categories of polymers, a heat quantitatively precisely measured and evenly distributed within an active matrix allows the polymer network to be temporarily deconstructed in order to accelerate the release of any type of solid and liquid active substances. The intra-matrix temperature reached during heating must not exceed the denaturing temperature of the incorporated active substance. This fact is exploited by the Applicant who has associated it with a pre-programmed electronic control card in order to obtain a release of a determined and sufficient quantity of active substance conferring on a given treatment a better effectiveness depending on the intended target. The controlled release profile of the active substance obtained thanks to the programming of said card constitutes the basic reference profile.

According to the invention, in order to achieve even heating of said matrix, the heating element is embedded in the active matrix. The heating element can also be over-molded directly onto said matrix and may also fit into the latter; in the latter case, it can be removable.

Given that the temporary expansion of the polymer network and the increase in vaporizing pressure of the active substance itself encourage its release from the active matrix, the Applicant found that embedding at least one heating element in the active matrix constitutes an efficient means of ensuring even intra-matrix heating so that heating by Joule effect causes the release of the active substance. The number of heating elements that must be present or the geometry of said heating element takes into account the dimensions of the active matrix to be heated, as well as the physico-chemical properties of the active substances namely the denaturing and boiling temperatures, the thermal properties of the matrix namely their thermal conductivity and the temperatures of degradation and softening of the polymers. In order to promote the acceleration of release of the active substance, preference is given to an openwork geometric form or any other design that increases the exchange surface of the matrix.

According to the invention, the electricity generator is necessarily connected to the electronic control card that controls the electricity supply from said generator to the heating element, plus the other electrical components of the system, including in particular the fan(s) and collectors; said card being pre-programmed. This programming involves varying, in a manner dependent upon one another or otherwise, three essential parameters, namely heating intensity, heating time and heating frequency depending on the composition of the matrix, the physico-chemical properties of the active substance and above all the necessary and sufficient quantity that has to be released in order to confer on a given treatment a better effectiveness. Heating intensity is directly proportional to the power of the electricity generator. The necessary and sufficient quantity of active substance is predetermined and known for example via experiments, laboratory tests, existing products available on the market that are widely recognized or even via literature. Thus, once a significant change in behavior, a significant physiological change or a significant environmental change compared to the reference values is reported, the control function of the electronic card will automatically decide, depending on the instructions defined by the algorithm, to trigger the activation of the heating element, the frequency of activation and the duration of activation, these three parameters being linked together or not.

In fact, said card controls the activation of the heating element by supplying the latter with electricity causing the matrix to be heated, which triggers the release at controlled and modulatable speed of the active substance. Heating occurs repetitively or sequentially so that the matrix releases almost all of the active substance. At the end of each heating, it is sometimes preferable to leave the matrix to resume its original behavior by leaving it to cool before reactivating it by another heating. In certain cases, in order to obtain a continuous release of active substance, it is preferable to reactivate the matrix successively and sequentially by heating without leaving it to cool down to ambient temperature.

According to the invention, the electronic control card is programmed to start, stop and regulate the power of the electricity generator. Heating is systematically stopped either when the intra-matrix temperature measured by the temperature probes is around 2° C. to 5° C. below the denaturing temperature of the active substance or that of the matrix, or when it is equal to the setpoint temperature previously indicated during programming.

According to an embodiment of the invention, the duration of a heating period is adapted according to the dimensions, thermal characteristics, namely the thermal conductivity of the matrix, as well as the physico-chemical properties, namely the degradation or denaturing temperature of the active substance, and above all the essential dose to be released in relation to the target in the environment concerned. The duration of a heating period can vary for example between 3 seconds and 60 minutes or even several hours.

According to an embodiment of the invention, said generator is capable of developing an electrical power sufficient to raise the temperature of the matrix to the programmed temperature. The electrical power to be received by the active matrix takes into account the surface of said matrix to be heated as well as its dimensions. For example, a power of around 60 watts allows a piece of polymer with a volume of around 600 cm³ to be brought to 70° C. in 15 minutes. Said electrical generator is chosen from a battery pack, electric battery, fuel cell, generating set, solar panel or the public electricity network known to a person skilled in the art.

According to another embodiment of the invention, the electronic card is capable of managing, if necessary, the start-up, stoppage and power of a fan. Said fan is placed near the active matrix so as to promote the propagation of the active substance into the atmosphere and to generate a concentration gradient that can be essential in controlling certain pests.

According to another embodiment of the invention, in order to complete and optimize the reference release, the electronic card is connected to a signal collector via a line-based and/or wireless network. Said collector is a device capable of transforming physico-chemical and physiological values into electrical signals. Said values are for example temperature, pressure, pH, air, smoke, humidity, noise, luminosity, presence, movement, or a combination thereof. Said collector can for example be a sensor, a probe, a detector or a combination thereof. Depending on the signals, said card adapts the operation of the generator and therefore the conditions of activation of the heating elements.

The system according to the invention can be used in the treatment of a human target, where a person is to be equipped with several onboard sensors, particularly accelerometers placed, for example, in a knee orthosis to measure movements, and physiological sensors arranged, for example, in a belt encircling the torso to measure heart rates. To a part of the body is applied a transdermic adhesive patch with a polymer matrix loaded with a therapeutic composition comprising thermal probes, a heating element, a battery, the activation of which is controlled by an onboard electronic card, after fusion and processing of the stored data, said data having been gathered by actimetry and physiological sensors. During fusion, the value ranges both of the movements and heart rates above which battery operation is triggered, at a specific intensity and for a specific period, are programmed into the algorithm.

The collectors described above are connected to an electronic control box by means of a line-based and/or wireless communication network and are placed on the active matrix, in the active matrix or near the active matrix, in the environment that surrounds it or a combination thereof. According to the invention, the heating element is an electric heating resistor chosen from a sheathed heating wire, a nickel-chrome alloy or titanium alloy resistance wire, a resistor in the form of a plate, metal ball or glow rod or a combination thereof. In the case of a heating wire, the resistivity is for example between 1 and 100Ω.

According to an embodiment of the invention, the polymer matrix itself can be modified in order to improve its thermal conductivity. To do this, fine metal particles are added to the polymer matrix, which significantly influence its thermal conductivity so as to limit its thermal inertia and improve the diffusion speed. Thermal conductive particles include:
polymers with added conductive loads derived from carbon such as carbon black, carbon fibers or tubes and graphite particles,
metals such as aluminum, copper, metal pigments such as silver, nickel or copper, or alloys such as iron/copper, aluminum/copper and other combinations known to a person skilled in the art,
conductive ceramics.

In the case of a heating element in the form of a plate, the latter is composed of a very thermally conductive material capable of withstanding a maximum power of, for example, up to 15 watts/cm².

According to an embodiment of the invention, in order to optimize the diffusion of the heat generated by heating within the active matrix, heat dissipaters such as insulated metal substrates in the form of aluminum plates surmounted by a fine ceramic insulating layer, itself surmounted by a copper printed circuit, are connected to the heating elements. The dissipaters are directly connected to the electricity generator. Said dissipaters having the above-mentioned characteristics are conventional dissipaters well known to a person skilled in the art such as insulated metal substrate (IMS) dissipaters. Said dissipaters are connected, for example by welding, to said heating elements.

Heating by Joule effect generated by the heating element is capable of causing the release of the active substance incorporated in the active matrix without causing either chemical degradations or irreversible structural degradations of said matrix. In any case, when the active matrix is based on thermosetting polymers, the intra-matrix temperature reached on heating is below the degradation temperature of said thermosetting polymers. Said degradation temperature is below or equal to 200° C. When the active matrix is made of thermoplastic polymers, the intra-matrix temperature reached on heating is below the softening point of said thermoplastic polymers. Said temperature is below or equal to 180° C.

According to the invention, the active substance represents between 0.1% and 50% by weight in relation to the total weight of the active matrix. The active substance is preferably formulated by dispersing it in a compatible vector and/or solvent known to a person skilled in the art. Other additives such as plasticizers, catalysts, surfactants, stabilizers, bittering agents, colorants and various loads can be added when said liquid composition is being formulated. Said additives are those currently used by a person skilled in the art. The active substance can be liquid or solid (amorphous and crystalline).

The active substances have functions such as attractive like kairomones, repellent, insecticide, insect repellent, fungicide, larvicide, bactericide, rodenticide, perfuming, deodorizing, analgesic, therapeutic, cosmetic, phytosanitary or even biostimulating.

Insecticide/insect repellent substances are chosen from the compounds of the pyrethrinoid family and their derivatives, the pyrethrins, DEET (N-diethyl-3-methylbenzamide), Icaridine®, IR3535® (ethyl-3-acetyl(butyl)amino-propanoate), organochlorines, organophosphorates, organophosphates (e.g. Diazinon®, Chlorpyrifos®, carbamates (e.g. Propoxur®, Fipronil®, neonicotinoids, sulfones and sulfonates, formamidines, benzoylureas, essential oils, plant extracts, terpenic hydrocarbons and their derivatives, vegetable oils or a mixture thereof.

Examples of non-volatile or low-volatile substances include DEET, IR3535®, Icaridine®, citronellol and citriodiol whose boiling temperatures are 240° C., 292° C., 272° C., 225° C. and 268° C. respectively.

The attractive substances are chosen from semiochemical molecules, essential oils, plant extracts, vegetal oils and flavors.

The deodorizing substances are molecules of natural origin or synthesis chosen from essential oils, perfume essences, perfumes and plant extracts.

The therapeutic substances are chosen from drugs, essential oils and alkaloids.

In the case of thermoplastic polymers, the active substance is formulated in liquid composition thanks to the support of compatible liquid vectors to obtain a homogenous liquid active composition. Said liquid composition is then incorporated into polymer granules according to the teachings of patents FR2901172, FR2956345 and application FR14/02377 filed in the Applicant's name, incorporated herein by reference. The compatible liquid vectors used in the above-mentioned processes are those that are currently used and known to a person skilled in the art.

In the case of thermosetting polymers, the active substance is formulated in liquid composition in the same way as for thermoplastic polymers. Said liquid active composition is then mixed at ambient temperature with the liquid solution of the polyol phase complex according to the teachings of patent application FR2992325 filed in the Applicant's name, incorporated herein by reference.

According to the invention, the matrix is manufactured from synthetic or biosourced thermoplastic polymers, biodegradable or otherwise, or from thermosetting polymers.

The thermoplastic polymers are of fossil origin. They are chosen from the copolymers of ethylene and vinyl acetate whose vinyl acetate content is between 15% and 60%, the polyester- or polyether-based thermoplastic polyurethane (TPU) elastomers, absorbent-grade copolyamides and polyamides (PA) such as PA6, PA10, PA12, absorbent-grade polyether block amides, absorbent-grade low-density polyethylenes (LDPE), starch-grafted polyethylenes, polyvinyl chlorides and their copolymers, polyesters, styrenic polymers such as SEBS, SIS or a mixture thereof.

The biodegradable polymers are biopolymers extracted directly from the biomass and their derivatives, or polyesters obtained by bacterial fermentation or by polymerization of monomers resulting from the biomass, or fossil resources, as well as the combination of these three groups. These include biopolymer derivatives such as cellulose acetate and polyesters such as polybutylene-adipate-co-terephthalate. The combination of polylactic acid and polyhydroxybutyrate can also be used or even the combination of an agro-polymer and a polyester such as starch and polybutylene-adipate-co-terephthalate, such as cornstarch and polylactic acid or a mixture of all of the above-listed biodegradable polymers.

The thermosetting polymers are reticulated polyurethanes obtained from a polyol resin that is liquid or rendered liquid and an isocyanate that is liquid or rendered liquid then formed by casting. Advantageously, the choice of the two essential components, namely polyol resin and isocyanate is adapted to suit the desired mechanical property of the matrix as well as its aptitude to minimize the release of the active substances incorporated in the matrix.

The isocyanate is chosen from isocyanates with at least 2 functions, with an aromatic or aliphatic structure. These include toluene diisocyanate (TDI), hexamethylene diisocyanate (HDI), methylene diphenyl diisocyanate (MDI), dicyclohexylmethane diisocyanate (H12MDI), isophorone diisocyanate (IPDI), naphthalene diisocyanate (NDI), O-tolidine diisocyanate (TODI), para-phenylene diisocyanate (PPDI) and their prepolymers.

The polyol resin is chosen from that having at least 2 hydroxyl functions, with a long chain or short chain, based on polyesters, polyethers, polythioethers, polyacetals, polycarbonates, polyesteramides, naturally hydroxylated or modified vegetable oils or a mixture thereof. The polyols can be replaced by polyamines having at least 2 amine functions, chosen from aliphatic or aromatic polyether amines.

According to the invention, the polymer matrix is formed according to plastics processing techniques well known to a person skilled in the art.

Other subject matter of the present invention relates to a method for effectively treating targets, in the form of a human being, an animal, insects or a zone delimited in an open or closed environment, located in the same place or in spatially different places, by means of at least one active substance, the sequential or continuous diffusion of a pre-determined controlled quantity of which solely at precise appropriate moments has an appropriate beneficial effect corresponding to the nature of the treatment of said targets, said method involves using the system according to claim 1.

According to an embodiment, when the active matrices are multiple and distributed in different places or installed in series, in such a case, on the one hand, each heating element of each of the matrices is connected to a single and common electronic control card and is arranged within a single central location serving as an electronic box from which said electronic card controls, individually, the activation of each heating element, depending on the data collected from the collectors and, on the other hand, the electronic card itself is connected either by a carrier electric cable, itself connected to a mains generator, or connected to a battery.

In fact, heating is activated sequentially. As an activation sequence is formed by the sum of the heating time and the rest time of the active matrix, the heating time is variable, between 10 seconds and several hours. Thus, the method involves activating the active matrix sequentially or continuously for as many times as necessary until the matrix releases almost all of the active sub stance.

For a better understanding of the claimed subject matter, the following Figures are intended to provide a detailed picture of the preferred structure of the system as described in the present invention, as well as its mode of operation.

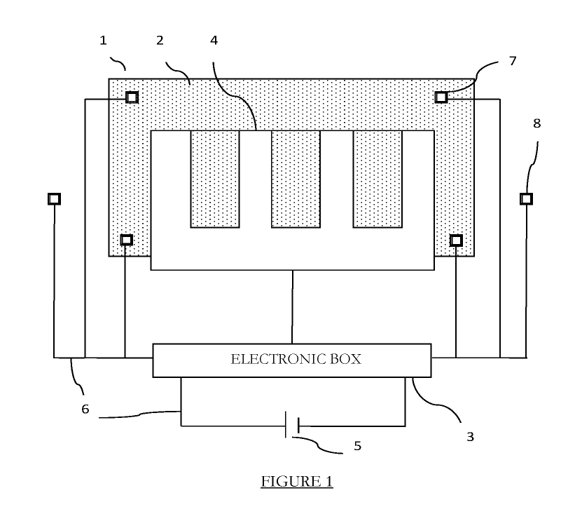

FIG. 1 Diagram of the matrix system according to the invention, comprising an active matrix, a heating element connected to an electric generator controlled by an electronic card housed in an electronic control box.

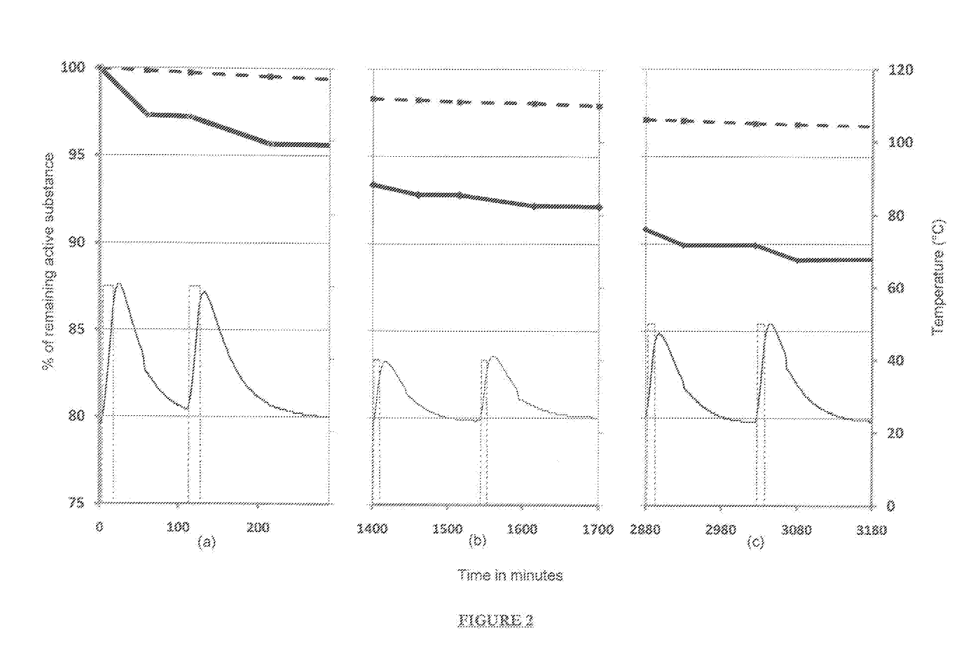

FIG. 2 Reference release profile of a peppermint essential oil obtained by the controlled matrix system with a thermosetting polymer matrix according to the invention.

FIG. 3 Reference release profile of a very low-volatile substance (citriodiol) obtained by the matrix system with a thermoplastic polymer matrix according to the invention.

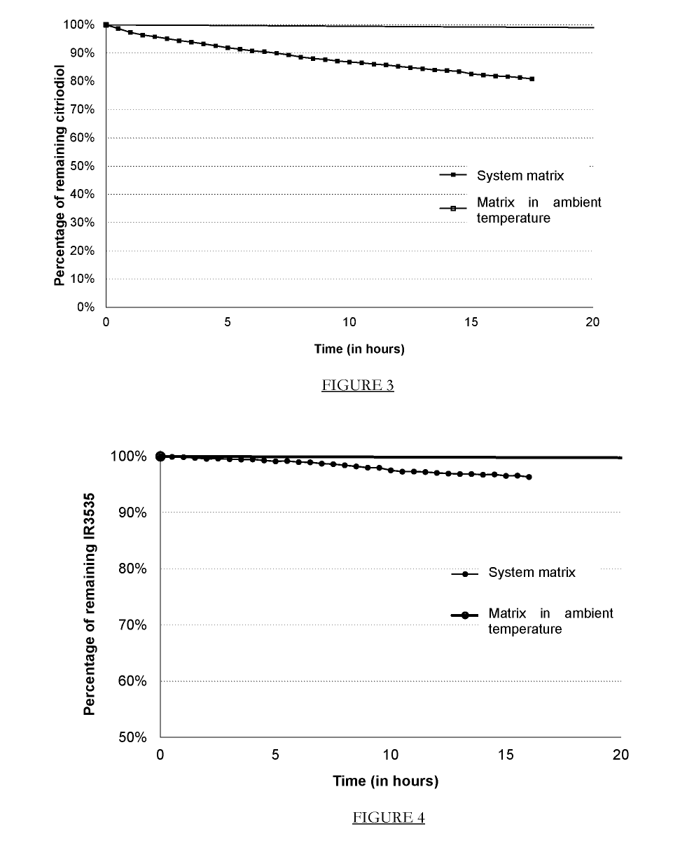

FIG. 4 Reference release profile of a non-volatile substance (IR3535®) obtained by the matrix system with a thermosetting polymer matrix according to the invention.

FIG. 1 represents an overall view of a prototype of the controlled matrix system (1) for the controlled and modulatable distribution of an active substance according to the invention. The system (1) is formed by an active matrix (2) in the form of a pin with five teeth. Said active matrix (2) can be cylindrical, polyhedral or spherical in shape. The heating element (4) also in the form of a pin with four teeth is embedded in the active matrix (2) so as to uniformly heat said matrix (2) by Joule effect. According to a variation of the invention, in order to optimize the dissipation of the heat generated by the heating element (4) inside the matrix (2), a heat dissipater (not shown) can be associated with said heating element (4). In this case, the dissipater is directly connected to the generator (5) or welded to the heating element (4).

The heating element (4) is connected to the electronic control box (3) which houses an electronic card (not shown) pre-programmed in terms of heating time, heating intensity and heating frequency in order to control it so as to obtain an active substance reference release profile. Said electronic control box (3) is connected to an electricity generator (5) by means of a line-based network (6). In order to optimize the reference release, temperature probes (7) located on the matrix (2) measure the intra-matrix temperature. Similarly, the electronic card adapts the activation of the heating element (4) on receiving signals transmitted by the collectors (8) to the electronic box (3). This is the case, for example, of multiple or series-mounted active matrices (2) placed individually in spatially different locations where the collectors (8) are movement detectors. Heating stops when the temperature measured by at least one of the heat sensors (7) is almost, between 2° C. and 5° C., below the denaturing temperature of the active substance or the pre-set setpoint temperature. One or more heating elements (4) are embedded in the active matrix (2) in order to uniformly heat it. The box (3) is connected to the generator (5) by means of a conventional line-based network.

According to a variation of the invention, the matrix system (1) is mounted in series. In this case, the electronic control box (3) is connected simultaneously to several different heating elements (2) embedded in different active matrices (2). Electric switches can then be placed within the electrical assembly connecting said electronic box (3) and each of the heating elements (2). In this way, the heating of the active matrices (2) also mounted in series, is controlled by a single electronic control box (3).

An in-series assembly like the one described above is advantageous when seeking to reach targets located in spatially different places.

The controlled stand-alone matrix system according to the invention has several advantages. It is capable of independently distributing active substances in controlled and modulatable quantities solely at precise appropriate moments while conferring to a given treatment a better effectiveness. In fact, thanks to the programming of the electronic card that controls the electrical supply to the electrical components by the generator, the user activates the system at will so as to trigger the release of the active substance according to a reference profile. In fact, it offers the possibility of increasing the intrinsic efficiency of an effective active substance at ambient temperature rather than by contact and which, by heating, comes to be by diffusion by volatility. The system according to the invention also allows the active matrix of almost all of the active substance to be emptied, unlike the conventional devices of passive diffusion at ambient temperature. Depending on the polymers used, it is possible to influence the mode of operation of the system which represents great flexibility and high adaptability of application. Lastly, when it is mounted in series, or when the matrices are multiple, the controlled matrix system is capable of simultaneously distributing one or more different active substances for several identical and/or different targets even if the latter are spatially far apart in relation to one another. In this case, one active matrix is stimulated independently of another to achieve better effectiveness.

The examples below serve to illustrate the matrix system according to the invention, without restricting its scope.

### EXAMPLE 1: CONTROLLED STAND-ALONE MATRIX SYSTEM WITH A THERMOSETTING POLYMER MATRIX

Previously, experiments conducted in the laboratories of the Applicant identified peppermint essential oil (PMEO) as an active substance having a significant repellent effect on houseflies (Musca domestica). To achieve this, a three-input olfactometer revealed the significantly sufficient quantities of PMEO required to achieve a repellent effect on flies. Table 1 below shows the results obtained.

TABLE 1
Repellent Effect of PMEO on Flies

| | Average flow rate | | | |
|---|---|---|---|---|
| | of PMEO (mg/hour) | | | |
| | 0 | 5.2 | 25.8 | 48.2 |
| Percentage of average time spent by the flies | 55% | 47% | 5.6% | 2.1% |
| in the presence of PMEO | | | | |

### Equipments

The following equipment was used:
a 1-liter open cylindrical reactor provided with a metal agitator terminating in a disk 6 cm in diameter, the agitator being operated by a speed-regulated motor,
a 24 V DC, 2.5 A, 60 W electric transformer providing the system's electrical supply;
2 PT100 heat sensors;
a sheathed heating wire with a resistance of 80 Ω/m for a total length of 25 cm. The two ends of the heating wire were directly connected to the electricity generator;
an electronic box that houses an electronic card supporting a calculator (microcontroller) that simultaneously manages the heating sequences, the power delivered by the transformer ranging between 0.1 and 60 W and the electrical interfaces. The sensors and heating wire were connected to the electronic box via a terminal block. The microcontroller was configured by a computer by means of a USB connection so that the supply of active matrix was performed by programmed activation sequences.
a polypropylene mold whose imprint has an open cylindrical form 10 cm in diameter and 2 cm thick.

To manufacture the active matrix, the following inputs were used:
a 2-function MW=178.28 short-chain polyamine resin, sold under the brand name XL1701®;
a 3-function MW=4800 polyol resin, sold under the brand name G48®;
a 2-function prepolymer of toluene diisocyanate (TDI) with an NCO percentage of 6.1, sold under the brand name LU-T95®;
a 2-function MDI prepolymer (diphenylmethylene diisocyanate) with an NCO percentage of 24.5, sold under the brand name SUPRASEC 2029®;
peppermint essential oil (PMEO) as the active substance with repellent action against flies;
solvent that is 2-ethylhexyldiphenyl phosphate, sold under the brand name SANTICIZER® 141;
a red dye in the form of fine powders sold under the brand name Rouge au Gras W320®.

All of the following operations are performed at ambient temperature.

### Preparation of the Liquid Composition Containing PMEO

18.3 g of PMEO, 51.2 g of SANTICIZER® 141 and 0.1 g of dye are placed in a beaker then mixed together under low agitation. After 5 mn of agitation, an active composition is obtained in the form of a homogenous liquid mixture.

### Preparation of the Polyol Liquid Mixture

13.2 g of XL1701® resin in liquid form, together with the liquid active composition obtained above, are then placed in the reactor. Everything is mixed by agitation at 1300 rpm for 30 seconds. A homogenous liquid mixture called “polyol phase complex” is obtained.

100 g of diisocyanate LU-T95® is added to the polyol phase complex obtained, then this is agitated at 1300 rpm for 30 seconds. A homogenous liquid mixture of polyurethane ready for casting is obtained.

### Shaping the Cast Polyurethane Active Matrix

The homogenous liquid mixture of polyurethane is cast in the above-described cylindrical mold. When the mixture fills the mold to mid-height, the heating wire is arranged therein in a zigzag pattern with the two ends over handing so that they can be connected to the generator. At the same time, the 2 heat sensors are equally spaced on the matrix undergoing polymerization. Then, the rest of the liquid mixture of polyurethane is cast to the desired dimensions. The heating wire as well as the heat sensors are thus embedded in the active matrix. This assembly is then left so that polymerization can be completed. After about 3 minutes, the part is removed from the mold. A cylindrical part 10 cm in diameter and 2 cm thick of polyurethane matrix loaded with 10% PMEO by weight in relation to the total weight of the part is obtained. The two ends of the heating wire are then connected to the electric transformer, which is connected to the electronic box.

Note that the irreversible structural degradation temperature of the polyurethane matrix as it is formulated for this experiment is around 170° C. and the boiling point of PMEO is around 210° C.

Activation by Heating the Matrix Loaded with PMEO

In order to obtain the necessary quantities of PMEO to have an effective repellent effect against flies, the electronic card is programmed as follows:
systematic cut off of heating and thus of the generator when the intra-matrix temperature measured by the heat sensors is 150° C.;
activation of heating for 13, 7 and 9 minutes respectively, which corresponds to three activation sequences;
adjustment of heating for 110 sequences of activation so that the residual quantity of PMEO at the end of the last activation is less than 10% by weight in relation to the quantity initially incorporated;
adjustment of the power of the transformer to 30 watts, thus the intensity of heating;
deactivating heating when the measured intra-matrix temperature exceeds 22° C. in order to give the active matrix time to resume its original behavior.

A switch allows the electrical supply to the system to be cut.

The programming defined above provide a reference release profile that can be optimized by adding more signal collectors such as detectors located near the active matrices to detect the movement or presence of flies.

FIG. 2 represents the reference release profile of PMEO. It shows the curves of release by passive diffusion of PMEO from an unheated polyurethane matrix placed at ambient temperature (dotted-line curves) and the curves of release from a polyurethane matrix in which a heating wire is embedded (continuous thick lines). The curve in continuous thin lines shows the increase in intra-matrix temperature of the heated parts. The experiments described above were conducted on 3 different matrices (see Table 2).

The 3 heating times were (a) 13, (b) 7 and (c) 9 minutes respectively. The PMEO-loaded matrix was weighed after each heating period in order to determine the actual quantity of PMEO released. After each heating period, the matrix was left to rest, at room temperature, for a minimum of 40 minutes for the temperature to drop to 35° C. and 22° C. In parallel, a release kinetics was performed on an identical polyurethane matrix loaded with 10% PMEO by weight, but with no heating element. Said matrix slowly released PMEO by passive diffusion, the released quantity also being measured by differential weighing.

The intra-matrix temperatures measured were 60° C., 40° C. and 50° C. respectively for heating times of 13, 7 and 9 minutes.

Table 2 below summarizes the quantity of PMEO released from 3 polyurethane matrices. Each matrix weighed around 100 g for a quantity of PMEO of 10 g, i.e. 10% of the total weight of the matrix.

TABLE 2
Average quantities of PMEO released
from 3 cast polyurethane matrices

| | PMEO release flow rate based on activation time | | | |
|---|---|---|---|---|
| Polyurethane | No | 7 minutes of | 9 minutes of | 13 minutes |
| Matrices | activation | activation | activation | of activation |
| Matrix 1 (M1) | 10 mg/hour | 45 mg/hour | 65 mg/hour | 110 mg/hour |
| Matrix 2 (M2) | 9 mg/hour | 35 mg/hour | 50 mg/hour | 55 mg/hour |
| Matrix 3 (M3) | 6 mg/hour | 25 mg/hour | 40 mg/hour | 45 mg/hour |

### Composition of Matrix 1

The liquid solution forming the polyol phase comprised: 13.2 g of XL1701®, 18.3 g of PMEO, 0.1 g of dye, 51.2 g of SANTICIZER 141®;

The liquid solution of the isocyanate phase consisted of 100 g of LUT-95®.

### Composition of Matrix 2

The liquid solution forming the polyol phase comprised: 81.24 g of G48®, 10 g of PMEO, 0.1 g of dye and 5.12 g of SANTICIZER 141®.

The liquid solution forming the isocyanate phase consisted of 8.71 g of SS2029®.

### Composition of Matrix 3

The liquid solution forming the polyol phase comprised: 13.2 g of XL1701, 12.58 g of PMEO and 0.1 g of red dye.

The liquid solution of the isocyanate phase consisted of 100 g of LUT-95®.

Note that if a repellent effect of 5.6% is desired, which corresponds to a PMEO flow rate of 25.8 mg/h, the system according to the invention enables several possibilities to be adopted, in particular Matrix M2/7 minutes or even Matrix 3/7 minutes. Similarly, in order to obtain a repellent effect of 2.1%, Matrix M1/9 minutes, Matrix M2/9 minutes or even M2/13 minutes can in particular be used.

Note also that the system according to the invention enables a PMEO flow rate to be obtained that is controlled solely at precise appropriate moments and in sufficient quantity to significantly repel flies. After 110 sequences of activation lasting 13 minutes, the quantity of residual PMEO measured in Matrix M1 is 3% in relation to the quantity initially incorporated.

### EXAMPLE 2: CONTROLLED STAND-ALONE MATRIX SYSTEM WITH A MATRIX OF THERMOPLASTIC POLYMERS OF FOSSIL ORIGIN

The same materials are used as in Example 1 (electric transformer, heat sensors, programmed electronic card).

An ARBURG injection-molding machine is used, on which is mounted a mold bearing the imprint of an oval medallion.

A Heidolph heating plate (MR HEI-Standard) is used having a power flux density of 4.84 watts/cm². Said heating plate is embedded in the medallion which allows said medallion to be uniformly heated.

Granules of copolymers of ethylene and vinyl acetate (EVA) are used, sold under the brand name EVA538® and EVA461®, as well as citriodiol as an insect repellent substance.

### Preparation of the Liquid Composition Containing the Active Substance

13.5 g of citriodiol and 2 g of coprah oil are mixed in a beaker. After 5 mn of low agitation, a homogenous liquid mixture is obtained.

### Incorporation of the Liquid Composition of Citriodiol in the EVA Granules

67.5 g of EVA538® granules are placed in the glass reactor previously pre-heated to 70° C.;
the liquid composition is added drop by drop;
everything is agitated until all of the liquid is incorporated into the granules;
the reactor is cooled to 40° C. then 17 g of EVA461® granules are added during low agitation;
everything is cooled to 20° C. then the granules loaded with 13.5% of citriodiol are recovered.

### Shaping the Active Matrix

The loaded EVA granules of citriodiol obtained above are shaped into a 9 mm thick oval medallion with a surface area of 20 cm² and weighing around 10 g.

Two heat sensors are inserted into the medallion obtained above in order to measure the intra-matrix temperature during heating.

Activation by Heating the Medallion Charged with Citriodiol

The electronic card is programmed as follows:
systematic cut off of heating and thus of the generator when the intra-matrix temperature measured by the heat sensors is 60° C.;
activation of heating every 10 minutes followed by a 20-minute pause, which corresponds to an activation sequence lasting a total of 30 minutes;
adjustment of heating for 36 sequences of activation;
adjustment of the power of the transformer to 30 watts, thus the intensity of heating;
inactivation of heating when the measured intra-matrix temperature exceeds 22° C. in order to give the active matrix time to resume its original behavior.

FIG. 3 shows the reference release profile of citriodiol. This Figure shows the curves of release by passive diffusion of citriodiol from an unheated EVA matrix placed at ambient temperature (continuous-line curve) and the release curve corresponding to an identical matrix in EVA in which is embedded a heating plate (continuous-line curve on which each activation is represented by a square) according to the system covered by the invention. Sequential and successive activations are performed for 18 hours at a rate of 10 minutes of heating followed by 20 minutes' pause. The intra-matrix temperature measured is 45° C.

The system according to the invention thus allows the release of up to 20% by weight of the citriodiol initially incorporated after 18 hours. By contrast, the same matrix releases practically nothing at ambient temperature. Moreover, the system allows an average quantity of citriodiol of 5 mg/hour to be released solely at precise moments of the activation.

Lastly, after 185 sequences of heating, and at the end of the last heating period, the residual quantity of citriodiol measured is 9% by weight in relation to the quantity initially incorporated.

### EXAMPLE 3: CONTROLLED STAND-ALONE SYSTEM WITH A THERMOSETTING POLYMER MATRIX IN WHICH THE ACTIVE SUBSTANCE IS IR3535®

The same materials are used as in Example 1, with the exception of the heating wire which is replaced by a heating plate as in Example 2.

For the matrix, the same inputs are used as in Example 1. The PMEO is replaced by insecticide (IR3535®), which represents 30% by weight in relation to the total weight of the matrix.

The electronic card is programmed as in Example 2 except that the heating is systematically cut off when the intra-matrix temperature reaches 150° C.

FIG. 4 represents the reference release profile of IR3535® which is a non-volatile substance at ambient temperature. This Figure shows the curves of release by passive diffusion of IR3535® from an unheated cast polyurethane matrix placed at ambient temperature (continuous thick line curve) and the release curve corresponding to an identical matrix in cast polyurethane in which is embedded a heating plate (continuous-line curve on which each activation is represented by a circle) according to the system covered by the invention. The sequential and successive activations were performed for 18 hours at a rate of 10 minutes heating followed by 20 minutes' pause. The intra-matrix temperature measured was 45° C.

The system according to the invention thus allows the release of up to 3% by weight of IR3535® initially incorporated after 18 hours. By contrast, the same matrix releases absolutely nothing at ambient temperature even after 20 hours. Moreover, the system allows the release of an average quantity of IR3535® of 3.5 mg/hour solely at precise moments of activation.

After 590 sequences of heating, and at the end of the last heating period, the residual quantity of IR3535® measured is 9.5% by weight in relation to the quantity initially incorporated.

1. A controlled stand-alone matrix system for the controlled and modulatable distribution solely at precise appropriate moments of at least one active substance, the system comprising:
a non-layered polymer matrix loaded with said active substance, said matrix comprising at least one temperature probe,
at least one heating element embedded in said loaded matrix, said heating element being capable of heating evenly the entire said matrix so as to temporarily expand the polymer network in order to trigger the release of said active substance, and
an electricity generator connected to an electronic control card, itself connected to said heating element, whose supply of electricity by said generator is controlled by said electronic control card housed in an electronic box, said electronic control card being connected, also, to said at least one temperature probe.

2. The controlled stand-alone matrix system according to claim 1, wherein the programming of said electronic control card involves varying any one of heating intensity, heating time and heating frequency, wherein said parameters are dependent or independent upon one another.

3. The controlled stand-alone matrix system according to claim 1, wherein said electronic control card executes the functions chosen from:
management of starting and stopping an electrical supply to the heating element by a generator;
adjustment of the power developed by said generator;
adjustment of the duration, intensity and frequency of heating;
management of activation sequences;
management of the total duration of a treatment;
management, if necessary, of starting, stopping and the power of a fan.

4. The controlled stand-alone matrix system according to claim 1, wherein heating is systematically stopped either when the intra-matrix temperature is around 2° C. to 5° C. below the denaturing temperature of the active substance and/or that of the matrix, or when it is equal to a setpoint temperature previously indicated during programming.

5. The controlled stand-alone matrix system according to claim 1, wherein the electronic control card controls the starting or stopping of the supply of electricity to the heating element by the generator, based on the signal sent by at least one collector via a line-based and/or wireless communication network.

6. The controlled stand-alone matrix system according to claim 1, wherein heating is performed sequentially or continuously until the residual quantity of the active substance in the active matrix is less than 10%, preferably between 0.1% and 5% by weight in relation to the quantity initially incorporated.

7. The controlled stand-alone matrix system according to the claim 6, wherein the duration of a heating period varies between ten seconds and several hours.

8. (canceled)

9. The controlled stand-alone matrix system according to claim 1, wherein said electronic control card is connected to a signal collector capable of transforming physico-chemical values into electrical signals, said physico-chemical values are chosen from temperature, pressure, pH, air, smoke, humidity, noise, movement, luminosity or a combination thereof.

10. The controlled stand-alone matrix system according to claim 9, wherein said signal collector is chosen from a sensor, a probe, a detector or a combination thereof.

11. The controlled stand-alone matrix system according to claim 1, wherein said heating element is overmolded directly onto said matrix or fits into said matrix.

12. The controlled stand-alone matrix system according to claim 1, wherein said heating element is an electric heating resistor chosen from a sheathed heating wire, a nickel-chrome alloy or titanium alloy resistance wire, a resistor in the form of a plate, metal ball or glow rod or a combination thereof.

13. (canceled)

14. The controlled stand-alone matrix system according to claim 1, wherein the active substance represents between 0.1% and 50% by weight in relation to the total weight of the active matrix.

15. The controlled stand-alone matrix system according to claim 1, wherein said active substance has a function chosen from an attractant, a repellent, an insecticide, an insect repellent, a fungicide, a larvicide, a bactericide, a rodenticide, a perfume, a deodorant, an analgesic, a therapeutic, a cosmetic, a phytosanitary or a biostimulant.

16. The controlled stand-alone matrix system according to claim 15, wherein the insecticide or insect repellent substance is chosen from the compounds of the pyrethrinoid family and their derivatives, the pyrethrins, DEET (N-diethyl-3-methylbenzamide), Icaridine®, (ethyl-3-acetyl(butyl)amino-propanoate) IR3535®, organochlorines, organophosphorates, organophosphates, carbamates, neonicotinoids, sulfones and sulfonates, formamidines, benzoylureas, essential oils, plant extracts, terpenic hydrocarbons and their derivatives, vegetable oils or a mixture thereof.

17. The controlled stand-alone matrix system according to claim 1, wherein the active matrix is manufactured from synthetic or biosourced thermoplastic polymers, biodegradable or otherwise, or from thermosetting polymers.

18. The controlled stand-alone matrix system according to claim 1, wherein the active matrix is manufactured from thermosetting polymers.

19. The controlled stand-alone matrix system according to claim 17, wherein the thermoplastic polymers are chosen from the copolymers of ethylene and vinyl acetate whose vinyl acetate content is between 15% and 60%, the polyether- or polyester-based thermoplastic polyurethane (TPU) elastomers, absorbent-grade polyamides, absorbent-grade polyether block amides, absorbent-grade low-density polyethylenes, starch-grafted polyethylenes, polyvinyl chlorides and their copolymers, polyesters, styrenic polymers such as SEBS, SIS or a mixture thereof.

20. The controlled stand-alone matrix system according to claim 18, wherein the thermosetting polymers are reticulated polyurethanes obtained from a polyol resin that is liquid or rendered liquid and an isocyanate that is liquid or rendered liquid.

21.-22. (canceled)

23. A method for effectively treating targets, in the form of a human being, an animal, insects or a zone delimited in an open or closed environment, located in the same place or in spatially different places, by means of at least one active substance, the sequential or continuous diffusion of a pre-determined controlled quantity of which solely at precise appropriate moments has an appropriate beneficial effect corresponding to the nature of the treatment of said targets, said method involves using the system according to claim 1.

24. The method according to claim 23, wherein when the active matrices are multiple and distributed in different places or installed in series, each heating element of each of the matrices is connected to a single and common electronic control card and is arranged within a single central location serving as an electronic box, from which said electronic card controls, individually, the activation of each heating element (4), depending on the data collected from the collectors, said electronic control card itself being connected either by a carrier electric cable, itself connected to a generator (5), or connected to a battery.
